(19) United States
(12) Patent Application Publication
(10) Pub. No.: US 2005/0010990 A1
(43) Pub. Date: Jan. 20, 2005

(54) Four-fingered welding glove

(76) Inventor: Jong Cheol Jang, Dongsam-dong (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/762,117

(22) Filed: Jan. 20, 2004

(30) Foreign Application Priority Data
Jul. 18, 2003 (KR) 10-2003-0049981
Aug. 22, 2003 (KR) 10-2003-0058919
Aug. 22, 2003 (KR) 10-2003-0058920
Aug. 28, 2003 (KR) 10-2003-0060954

(51) Int. Cl. A41D 13/08; A61F 9/00

(52) U.S. Cl. 2/161.6

(57) ABSTRACT

A glove in which a thumb, an index finger, a middle finger, a third finger, and a little finger, or a thumb, an index finger, a middle finger, and a shell portion, are formed at one side of a main body and an opening portion for insertion of a hand is formed at the other side, which includes an integrated sheet in which a front index finger having a thumb hole formed at a lower end thereof, extending from a finger end, and having a wing formed at a side surface thereof, a rear index finger, a rear middle finger, and a rear shell portion, are formed, and a finger piece having a separated cut middle finger and a shell portion and sewn between the finger end and the wing of the front index finger.

### RELATED APPLICATIONS

This application is related to, and hereby incorporates by reference, the following patent applications:

U.S. Patent Application entitled “THREE-FINGERED WELDING GLOVE”, filed on even date herewith and having application Ser. No. ______ (Attorney Docket No. GRANP3.001AUS); and

U.S. Patent Application entitled “FIVE-FINGERED WELDING GLOVE”, filed on even date herewith and having application Ser. No. ______ (Attorney Docket No. GRANP3.003AUS)

### BACKGROUND OF THE INVENTION

1. Field of the Invention

The present invention relates to a four-fingered glove, and more particularly, to a four-fingered glove which is manufactured by sewing a specially cut finger piece in various shapes on a palm piece or an integrated sheet cut in various shapes, and separating the index finger and the middle finger and incorporating the third finger and the little finger into a shell portion, to form a glove used to protect a hand at a work site where a user holds or moves a sharp metal piece or hot metal or high temperature flames spout.

2. Description of the Related Technology

In general, a glove has a thumb, an index finger, a middle finger, a third finger, and a little finger, or a thumb, an index finger, and a shell portion, at one side and an opening portion for insertion of a hand is formed at the opposite side. The glove is manufactured of a thick leather material exhibiting a superior flame resistance effect to prevent damage or burning of a hand holding or moving a sharp or hot stuff according to the purpose of a work.

### SUMMARY OF CERTAIN INVENTIVE ASPECTS OF THE INVENTION

One aspect of the invention provides a four-fingered glove which is manufactured to include a thumb, an index finger, a middle finger, and a shell portion by forming a finger piece on an integrated sheet where a shell portion accommodating the third finger and the little finger together to secure a space between the fingers. Thus, the shell portion accommodates the third finger and the little finger only, which are not used well during work, rather than all the middle finger, the third finger, and the little finger together, so that the middle finger can freely moved during work and a fine work can be performed.

Thus, a pressing and hardening phenomenon is removed by removing a sewing line on the back of a hand or the outer side of a hand. A fine work, which has been performed inconveniently because a typical glove has a thumb, an index finger, and a shell portion, can be performed with a free hand motion by the present glove having a thumb, an index finger, a middle finger, and a shell portion. While another typical glove has an index finger portion, a middle finger portion, a third finger portion, and a little finger portion so that a space between fingers is narrow, in the present glove having an index finger portion, a middle finger portion, and a shell portion, the space between the fingers is relatively large so that the pressing and hardening phenomenon between the fingers is removed and a free motion of a hand is available. Consequently, a fatigue of hand is reduced and work efficiency is improved.

Another aspect of the invention provides a welding glove in which a thumb, an index finger, a middle finger, a third finger, and a little finger, or a thumb, an index finger, a middle finger, and a shell portion, are formed at one side of a main body and an opening portion for insertion of a hand is formed at the other side, the glove comprising an integrated sheet in which a front index finger having a thumb hole formed at a lower end thereof, extending from a finger end, and having a wing formed at a side surface thereof, a rear index finger, a rear middle finger, and a rear shell portion, are formed, and a finger piece having a separated cut middle finger and a shell portion and sewn between the finger end and the wing of the front index finger.

At least one rear stitch point between the rear index finger, the rear middle finger, and the rear shell portion is moved upward.

Another aspect of the invention provides a welding glove where a thumb, an index finger, a middle finger, a third finger, and a little finger, or a thumb, an index finger, a middle finger, and a shell portion, are formed at one side of a main body and an opening portion for insertion of a hand is formed at the other side, the glove comprising an integrated sheet in which a front shell portion having a thumb hole formed at a lower side thereof, extending from a finger end, and having a wing formed at a side surface thereof, a rear shell portion, a rear middle finger, and a rear index finger, are formed, and a finger piece having a separated cut middle finger and an index finger and sewn on the finger end.

Another aspect of the invention provides a welding glove where a thumb, an index finger, a middle finger, a third finger, and a little finger, or a thumb, an index finger, a middle finger, and a shell portion, are formed at one side of a main body and an opening portion for insertion of a hand is formed at the other side, the glove comprising a palm piece in which a thumb hole is formed at a lower side of a rectangle and a finger end is formed at an upper end thereof, a finger piece coupled to the palm piece, having a separately cut index finger, a middle finger, and a shell portion, and sewn on an upper end of the finger end, and a hand's back piece coupled to the palm piece and having a rear index finger, a rear middle finger, and a rear shell portion.

Another aspect of the invention provides a welding glove where a thumb, an index finger, a middle finger, a third finger, and a little finger, or a thumb, an index finger, a middle finger, and a shell portion, are formed at one side of a main body and an opening portion for insertion of a hand is formed at the other side, the glove comprising an integrated sheet having a front shell portion, in which a thumb hole is formed at a lower side thereof and a wing is formed at a side surface thereof, a front index finger having a wing formed at a side surface thereof, a rear index finger, a rear middle finger, and a rear shell portion, and a front middle finger piece which is separately cut and sewn between the wing of the front shell portion and the wing of the front index finger.

Another aspect of the invention provides a welding glove where a thumb, an index finger, a middle finger, a third finger, and a little finger, or a thumb, an index finger, a middle finger, and a shell portion, are formed at one side of a main body and an opening portion for insertion of a hand is formed at the other side, the glove comprising an integrated sheet in which a rear index finger, in which a thumb hole is formed at a lower side thereof, a rear middle finger, a front shell portion having a wing formed at a side surface thereof, and a front index finger having a wing formed at a side thereof, are integrally formed, and a front middle finger piece which is separately cut and sewn between the wing of the front shell portion and the wing of the front index finger.

### BRIEF DESCRIPTION OF THE DRAWINGS

The above and other features and advantages of embodiments of the present invention will become more apparent by describing in detail preferred embodiments thereof with reference to the attached drawings in which:

FIGS. 1A through 1D illustrate the back of a hand, the palm of a hand, the cutting pattern, and the 3-D shape of a typical welding glove;

FIGS. 2A through 2C illustrate the back of a hand, the palm of a hand, and the cutting pattern of another typical welding glove;

FIGS. 3A through 3D illustrate the back of a hand, the palm of a hand, the cutting pattern, and the 3-D shape of a four-fingered welding glove according to a first preferred embodiment of the present invention, which is formed into an integrated sheet;

FIGS. 4A through 4D illustrate the back of a hand, the palm of a hand, the cutting pattern, and the 3-D shape of the four-fingered welding glove according to the first preferred embodiment of the present invention, in which a rear side stitch point A on the integrated sheet is moved upward;

FIGS. 5A through 5D illustrate the back of a hand, the palm of a hand, the cutting pattern, and the 3-D shape of the four-fingered welding glove according to the first preferred embodiment of the present invention, in which a rear side stitch point B on the integrated sheet is moved upward;

FIGS. 6A through 6D illustrate the back of a hand, the palm of a hand, the cutting pattern, and the 3-D shape of the four-fingered welding glove according to the first preferred embodiment of the present invention, in which the rear side stitch points A and B on the integrated sheet are moved upward;

FIGS. 7A through 7D illustrate the back of a hand, the palm of a hand, the cutting pattern, and the 3-D shape of a four-fingered welding glove according to a second preferred embodiment of the present invention, which is formed into an integrated sheet;

FIGS. 8A through 8D illustrate the back of a hand, the palm of a hand, the cutting pattern, and the 3-D shape of a four-fingered welding glove according to a third preferred embodiment of the present invention, in which the front surface and the rear surface are separated from each other;

FIGS. 9A through 9D illustrate the back of a hand, the palm of a hand, the cutting pattern, and the 3-D shape of a four-fingered welding glove according to a fourth preferred embodiment of the present invention, which is formed into an integrated sheet; and

FIGS. 10A through 10D illustrate the back of a hand, the palm of a hand, the cutting pattern, and the 3-D shape of a four-fingered welding glove according to a fifth preferred embodiment of the present invention, which is formed into an integrated sheet.

### DETAILED DESCRIPTION OF CERTAIN EMBODIMENTS OF THE INVENTION

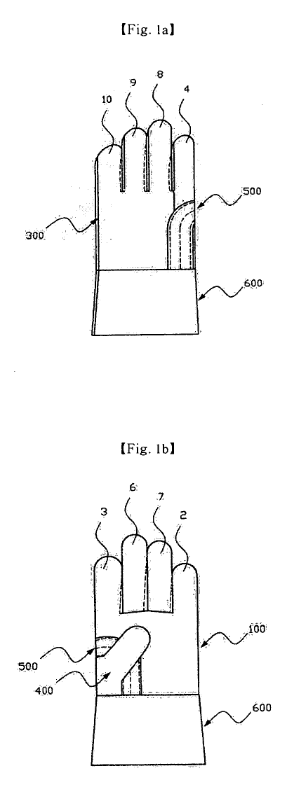

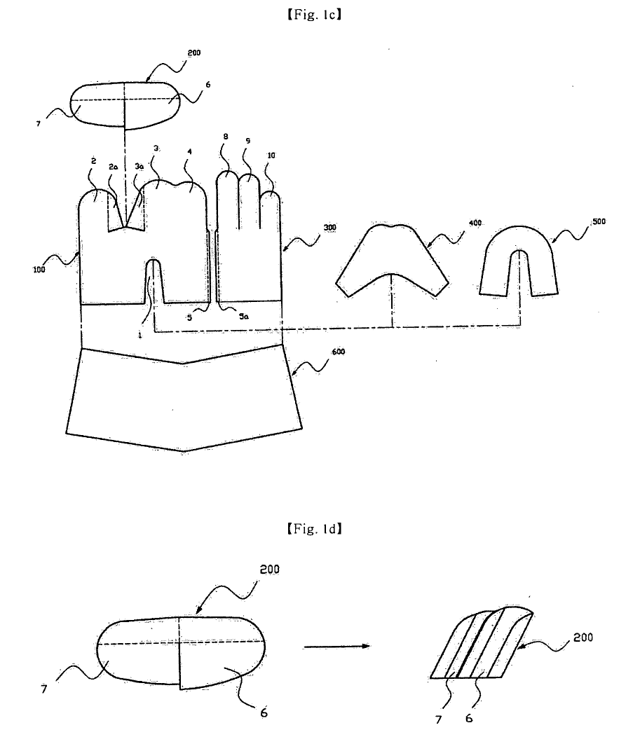

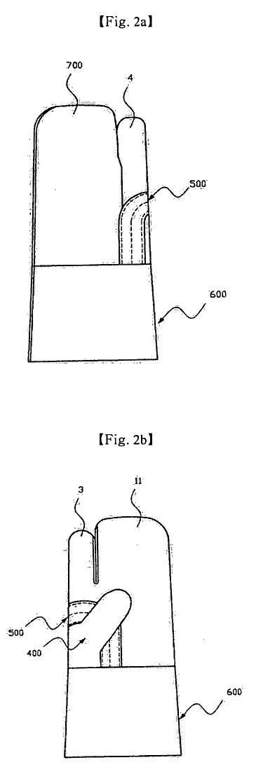

FIGS. 1A through 1D illustrate a typical welding glove. As shown in FIGS. 1A through 1D, a front little finger 2 having a thumb hole 1 formed at the lower end and a wing 2a formed at the upper end, an index finger 3 having a wing 3a, and a rear index finger 4, are integrally cut. A finger piece 200 having a middle finger 6 and a third finger 7 is separately cut on a front piece 100 in which a sewing end 5 is formed at one side end of the rear index finger 4. These are sewn between the front little finger wing 2a and the front index finger wing 3a. A rear middle finger 8, a rear third finger 9, and a rear little finger 10 are integrally formed. The sewing end 5 at one side end of the rear index finger 4 of the front piece 100 is sewn on a rear side piece 300 where a sewing end 5a is formed at one side end of the rear middle finger 8. A thumb piece 400 is sewn on the thumb hold 1 at the lower end thereof. A sewing line protection strap 500 is sewn on the upper surface of the thumb piece 400. A cuff 600 is sewn on the lower end.

In the above glove, to form a thumb, an index finger, a middle finger, a third finger, and a little finger, several thick cut pieces are sewn together so that the pressing and hardening phenomenon occurs by a sewing line sewn on the other pieces produces pain. Since the pressing and hardening phenomenon occurs between the fingers, for a long-time work, a user cannot wear the glove not fully but loosely or puts the glove off for some time, thus lowering work efficiency.

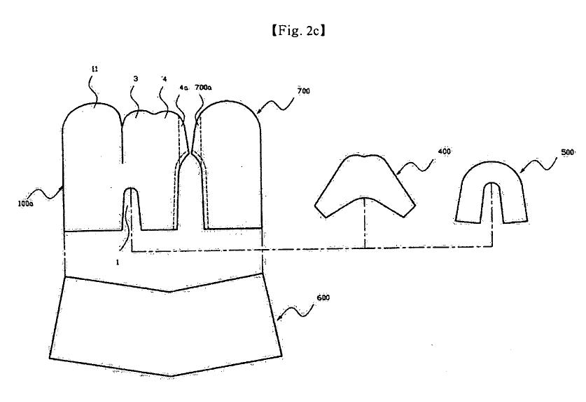

FIGS. 2A through 2C illustrate another typical welding glove. The glove includes a front piece 100a where the thumb hole 1 is formed at a lower end and a front shell portion 11, a front index finger 3, and a rear index finger 4 where a wing 4a is formed at a side surface thereof, are integrally cut, a rear shell piece 700 coupled to the front piece 100a and having a wing 700a formed at a side surface thereof, a thumb piece 400 sewn on the thumb hole 1 of the front piece 100a, and a cuffs 600 sewn on the front piece 100a and the lower end of the rear shell piece 700. The glove having the above structure is used at a work site where the middle finger, the third finger, and the little finger are not frequently used while the thumb and the index finger are mainly used. Since a space is sufficiently secured between the middle finger, the third finger, and the little finger, at a place where a work is performed for a long time, the pressing and hardening phenomenon between the fingers can be removed. However, since the number of fingers is small, it is difficult to pick or hold a small thing and work efficiency in a fine work is lowered.

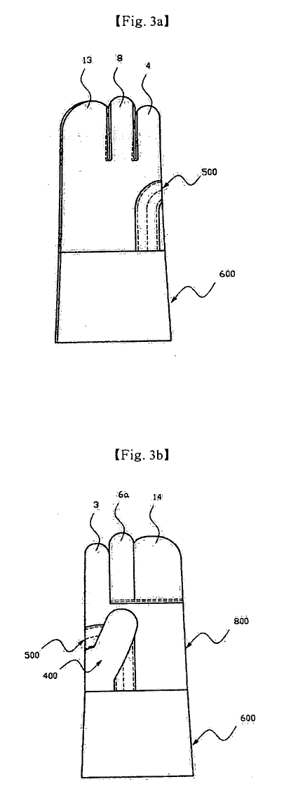

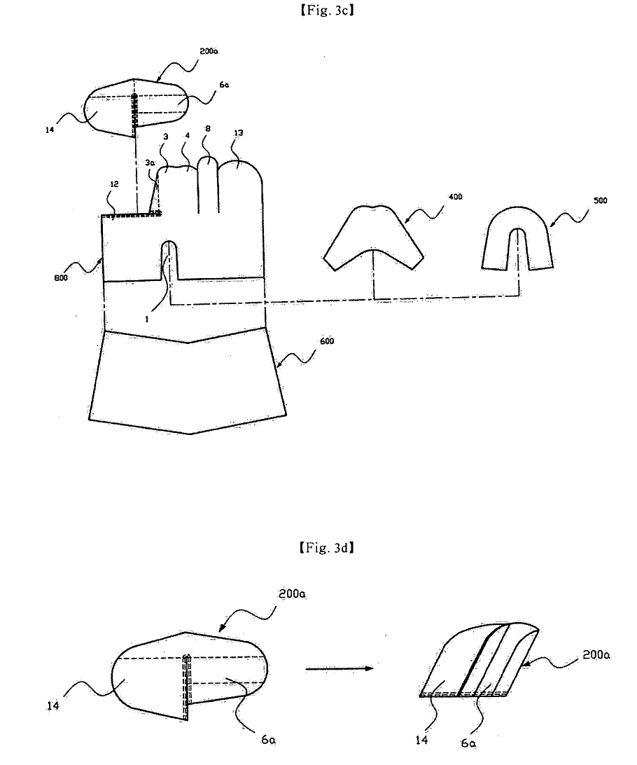

Referring to FIGS. 3A through 3D, a four-fingered glove according to a first preferred embodiment of the present invention is manufactured by sewing a finger piece 200a where a specially cut middle finger 6a and a finger shell portion 14 are formed, on an integrated sheet 800 where a front index finger 3 having a thumb hole 1 at a lower end, extending from a finger end 12, having a wing 3a formed at the side surface thereof, a rear index finger 4, a rear middle finger 8, and a rear shell portion 13 are formed, between the finger end 12 and the wing 3a of the front index finger 3, by being folded by a finger, sewing a thumb piece 400 on the thumb hole 1, and sewing a cuffs 600 at the lower end in a state in which the integrated piece 800 is sewn with the finger piece 200a.

In one embodiment of the present invention, the front index finger 3, the rear index finger 4, the rear middle finger 8, and the rear shell portion 13 are formed into the integrated sheet 800 and the front piece 100 and the rear piece 300 are sewn with the other piece, a sewing line is removed and a pressing and hardening phenomenon on the back of a hand is removed. In the typical glove where the index finger, the middle finger, the third finger, and the little finger are individually formed, a space is not present between the fingers so that the pressing and hardening phenomenon occurs between the fingers. In one embodiment of the present invention, however, the third finger and the little finger which are not used well during work are accommodated by the shell portion so that a space is provided between the fingers and the pressing and hardening phenomenon is removed. Also, although a sufficient space can be secured between the fingers in a glove accommodating all the middle finger, the third finger, and the little finger together, since the number of fingers to be accommodated is too large, motion of a hand is obtuse so that a fine work is difficult. However, in a glove according to one embodiment of the present invention, the third finger and the little finger which are not used well during work are accommodated in a space while the middle finger is freely used. Thus, a fine work is available.

A variety of gloves may be manufactured by cutting and sewing the integrated sheet 800 and the finger piece 200a, which will be described in detail based on the following preferred embodiments of the present invention.

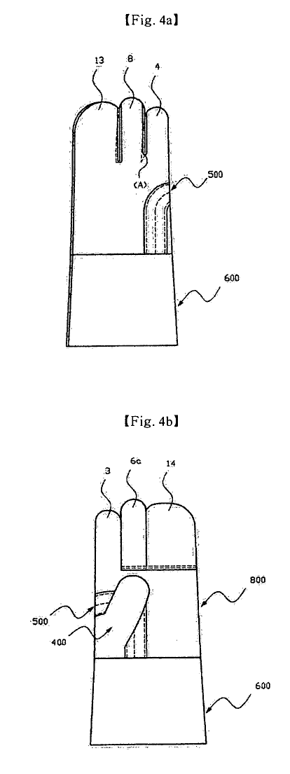

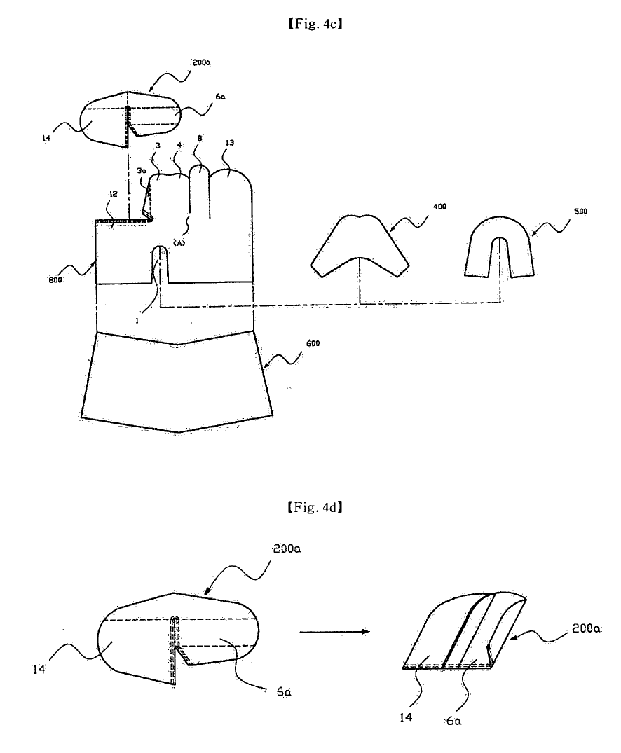

FIGS. 4 through 6 show a four-fingered glove according to the first preferred embodiment of the present invention, in which stitch points A and B are moved. In the drawings, the stitch point A is a stitch point on a rear surface between the rear index finger and the rear middle finger and the stitch point B is a stitch point on the rear surface between the rear middle finger and the rear shell portion.

As shown in FIGS. 4A through 4D, the front index finger 3 and the middle finger 6a of the integrated sheet 800 are bent at the same angle and the finger piece 200a is folded in a finger shape and sewn on the finger end 12 of the integrated sheet 800, to manufacture a glove.

Thus, since the rear stitch point A between the rear index finger 4 and the rear middle finger 8 moves over a rear surface of the first knuckle of a finger as much as the bending angle of the front index finger 3 of the integrated sheet 800 and the middle finger 6a of the finger piece 200a, the pressing and hardening phenomenon between the rear index finger 4 and the rear middle finger 8 is removed so that the glove wears conveniently and fatigue of a hand is reduced.

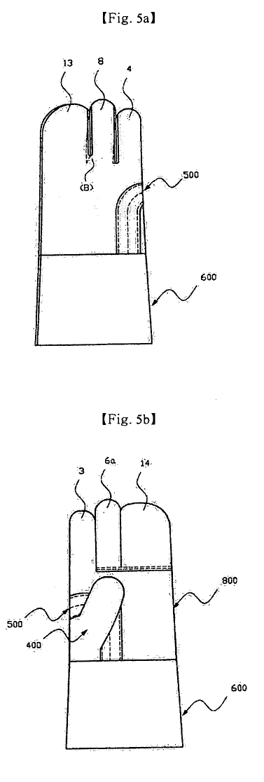

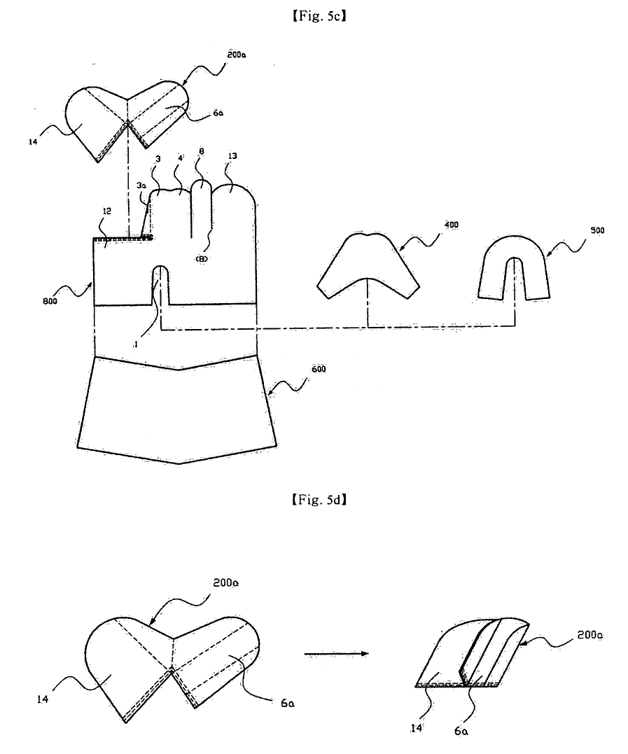

Also, as shown in FIGS. 5A through 5D, a portion between the middle finger 6a and the shell portion 14 is bent and formed to be open. The finger piece 200a is bent in a finger shape and is sewn on the finger end 12 of the integrated sheet 800, so that a glove is manufactured.

Thus, the stitch point B of the rear middle finger 8 and the rear shell portion 13 moved over the rear surface of the first knuckle of a finger as much as a portion between the middle finger 6a of the finger piece 200a and the shell portion 14 is bent and open. Thus, the pressing and hardening phenomenon between the rear middle finger 8 and the rear shell portion 13 is removed.

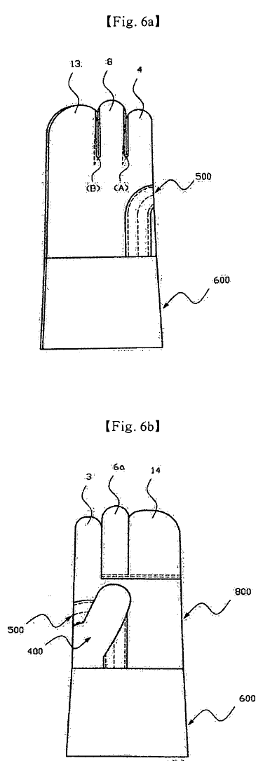

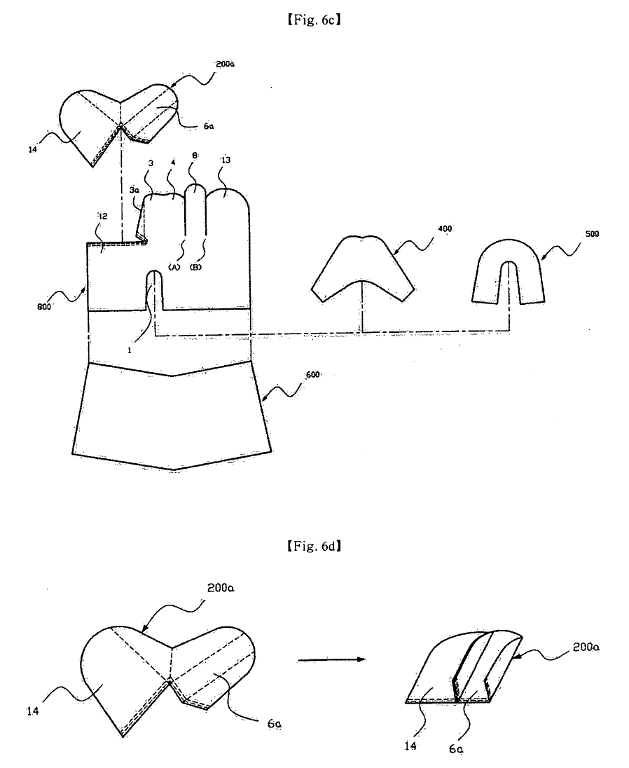

As shown in FIGS. 6A through 6D, the front index finger 3 and the middle finger 6a of the integrated sheet 800 are bent at the same angle. The middle finger 6a and the shell portion 14 is bent to be open. The finger piece 200a is folded in a finger shape and sewn on the finger end of the integrated sheet 800, so that a glove is manufactured.

Thus, the rear stitch point A between the rear index finger 4 and the rear middle finger 8 is moved over the rear surface of the first knuckle of a finger as much as the angle at which the front index finger 3 and the middle finger 6a of the integrated sheet 800 are bent. Simultaneously, the rear stitch point B between the rear middle finger 8 and the rear shell portion 13 is moved over the rear surface of the first knuckle of a finger as much as the front middle finger 6a and the shell portion 14 are bent and open. Thus, the pressing and hardening phenomenon between the rear index finger 4 and the rear middle finger 8, and the rear middle finger 8 and the rear shell portion 13, is removed.

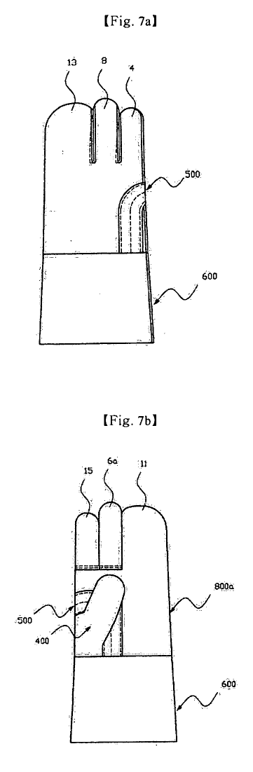

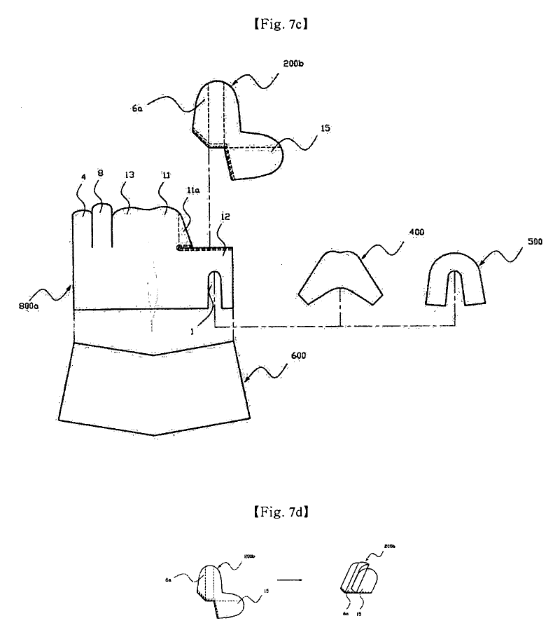

FIG. 7 shows a four-fingered glove according to a second preferred embodiment of the present invention. As shown in FIGS. 7A through 7D, a finger piece 200b where a specially cut middle finger 6a and the index finger 15 are formed is sewn with the finger end 12 on an integrated sheet 800a where the front shell portion 11, in which the thumb hole 1 is formed at the lower side and which extends from the finger end 12 and has the wing 11a formed at the side surface thereof, the rear shell portion 13, the rear middle finger 8, and the rear index finger 4 are formed.

In the glove according to the present preferred embodiment, the position of the portion where fingers are formed is moved in the integrated sheet described in the first preferred embodiment is moved and sewn. Thus, the sewing line formed on the outer side of a hand is formed at the side surface of the index finger so that, at a site where work is performed with the outer side of a hand contacting the floor, the pressing and hardening phenomenon on the outer side of a hand is removed. Also, the pain of a hand is alleviated and the pressing and hardening phenomenon is removed.

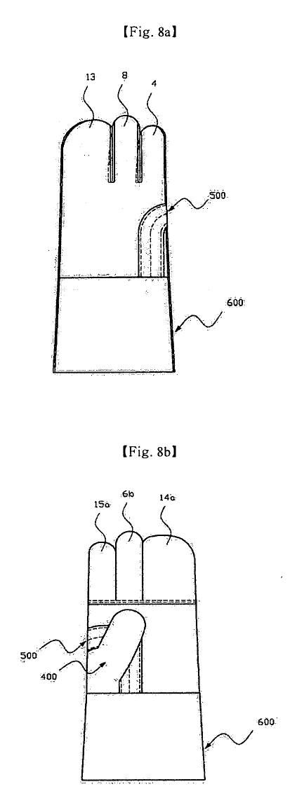

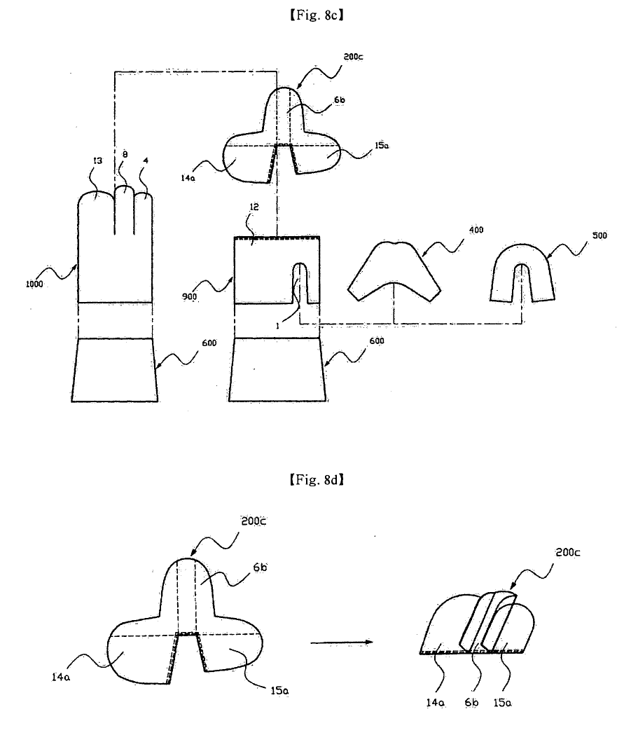

FIGS. 8A through 8D show a four-fingered glove according to a third preferred embodiment of the present invention, in which the front surface and the rear surface are separated. As shown in FIGS. 8A through 8D, the glove is manufactured by sewing a finger piece 200c where an index finger 15a, a middle finger 6b, and a shell 14a are formed by being separately cut, with a hand's back piece 1000 where the rear index finger 4, the rear middle finger 8, and the rear shell portion 13 are formed, on a palm piece 900 where the thumb hole 1 is formed at a lower side of a rectangle and the finger end 12 is formed at an upper side thereof.

Thus, the finger piece 200c folded in finger shape forms the whole of the index finger, the middle finger, and the shell so that the entire fingers are accommodated. Thus, a sufficient space is secured between the fingers and a free hand motion is available.

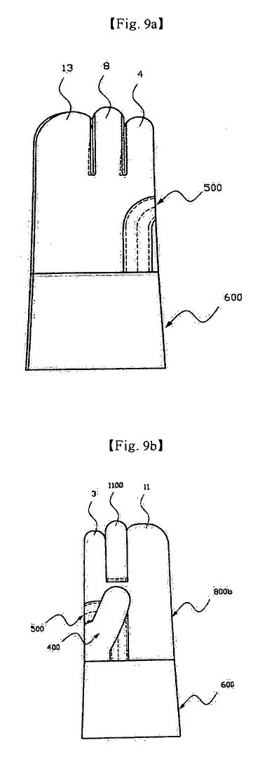

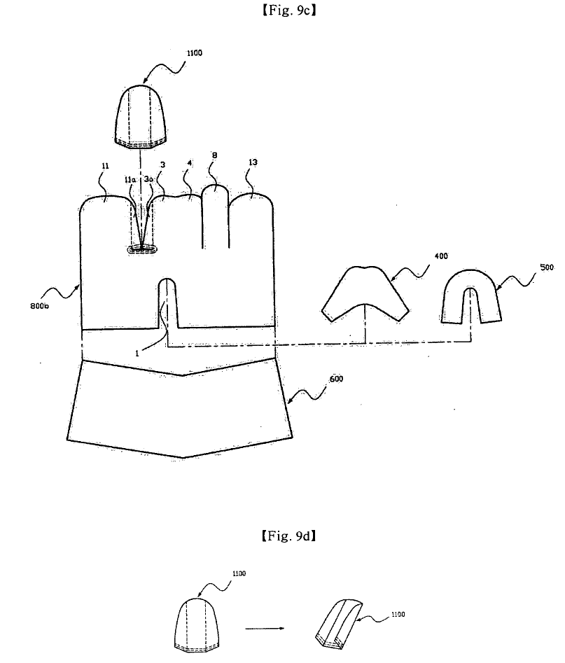

FIGS. 9A through 9D show a four-fingered glove according to a fourth preferred embodiment of the present invention. As shown in FIGS. 9A through 9D, the glove is manufactured by sewing a separately cut front middle finger piece 1100, which is folded in a case shape and inserted between the wing 11a of the front shell portion 11 and the wing 3a of the front index finger 3, on an integrated sheet 800b where the front shell portion 11 having the thumb hole 1 and the wing 11a formed at the lower and side surfaces thereof, respectively, the front index finger 3 having a wing 3a formed at the side surface thereof, the rear index finger 4, the rear middle finger 8, and the rear shell portion 13.

Thus, in the glove according to the present preferred embodiment, since only the front middle finger piece 1100 is inserted between the wing 11a of the front shell portion 11 and the wing 3a of the front index finger 3, and sewn, the glove is simply manufactured and efficiency in manufacturing is improved. Also, the sewing line is remarkably reduced so that the pressing and hardening phenomenon is remarkably reduced.

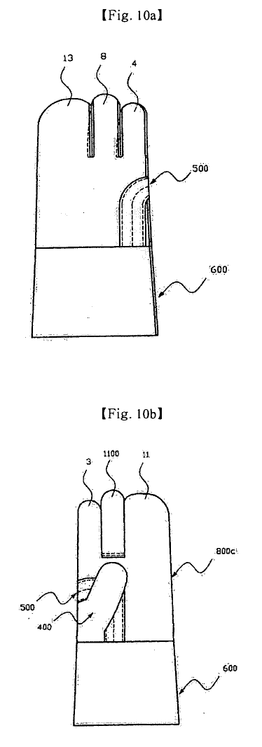

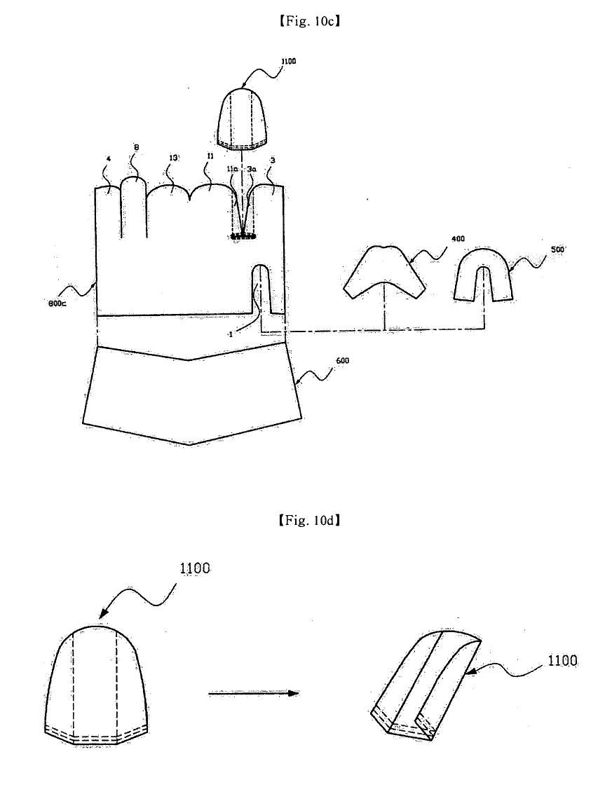

FIGS. 10A through 10D show a four-fingered glove according to a fifth preferred embodiment of the present invention. As shown in FIGS. 10A through 10D, the glove is manufactured by inserting the front middle finger piece 1100 between the wing 11a of the front shell portion 11 and the wing 3a of the front index finger 3 and sewing the same on an integrated sheet 800c where the rear index finger 4 having the thumb hole 1 formed at a lower side thereof, the rear middle finger 8, the rear shell portion 13, the front shell portion 11 having the wing 11a at the side surface thereof, and the front index finger 3 having the wing 3a formed at the side surface thereof.

In the glove according to the fourth preferred embodiment of the present invention, the position of a portion where the fingers are formed is moved. The sewing line formed on the outer side of a hand is moved to the side surface of the index finger. Thus, the pressing and hardening phenomenon on the outer side of a hand occurring at a site where work is performed with the outer side of a hand contacting the floor, is removed, so that pain in the hand is alleviated.

Although the present invention is described with respect to a welding glove, the whole scope of the present invention can be applied to any modification of a work glove wearing by removing the sewing line protection strap 500 and reducing the length of the cuffs 600.

As described above, in the four-fingered glove according to at least one embodiment of the present invention, since the glove has the index finger, the middle finger, and the shell portion, compared to the typical glove having the index finger, the middle finger, the third finger, and the little finger, a space between the fingers is secured. Also, since the middle finger is separately formed and the shell portion accommodating the third finger and the little finger is formed, compared to the typical glove in which the middle finger, the third finger, and the little finger are accommodated together, the number of the sewing lines is reduced and the pressing and hardening phenomenon is removed. Furthermore, since the middle finger is separated, a fine work is made easy, fatigue of the hand is reduced, and work efficiency is improved.

While this invention has been particularly shown and described with reference to preferred embodiments thereof, it will be understood by those skilled in the art that various changes in form and details may be made therein without departing from the spirit and scope of the invention as defined by the appended claims.

1. A four-fingered welding glove, comprising:
an integrated sheet including a front index finger portion, a rear index finger portion, a rear middle finger portion, a rear shell portion, a finger end, and a thumbhole;
a finger piece having a front middle finger portion and a front shell portion, wherein the finger piece is coupled to the finger end and the front index finger portion such that the front and rear index finger portions are together configured to receive an index finger, such that the front and rear middle finger portions are together configured to receive a middle finger, and such that the front and rear shell portions are together configured to receive a third finger and a little finger; and
a thumb piece coupled to the thumbhole of the integrated sheet and configured to receive a thumb.

2. The welding glove of claim 1, wherein the front index finger portion includes a wing at a side surface thereof, and wherein the finger piece is sewn on the finger end and the wing of the front index finger portion

3. The welding glove of claim 1, wherein a first stitch line is formed between the rear index finger portion and the rear middle finger portion, and a second stitch line is formed between the rear middle finger portion and the rear shell portion, and wherein one of the first and second stitch lines is shorter than the other one.

4. The welding glove of claim 1, wherein the front middle finger portion and the front shell portion are separately formed.

5. A four-fingered welding glove, comprising:
an integrated sheet having a front shell portion, a rear shell portion, a rear middle finger portion, a rear index finger portion, a finger end and a thumbhole;
a finger piece having a front middle finger portion and a front index finger portion, wherein the finger piece is coupled to the finger end and the front shell portion such that the front and rear index finger portions are together configured to receive an index finger, that the front and rear middle finger portions are together configured to receive a middle finger, and that the front and rear shell portions are together configured to receive a third finger and a little finger; and
a thumb piece coupled to the thumbhole of the integrated sheet and configured to receive a thumb.

6. The welding glove of claim 5, wherein the front middle finger portion and the front index finger portion are separately formed.

7. The welding glove of claim 5, wherein the front shell portion includes a wing at a side surface thereof, and wherein the finger piece is sewn on the finger end and the wing of the front shell portion

8. A four-fingered welding glove, comprising:
a palm piece having a finger end and a thumbhole;
a finger piece having a front index finger portion, a front middle finger portion and a front shell portion, coupled to the finger end of the palm piece;
a backhand piece having a rear index finger portion, a rear middle finger portion, and a rear shell portion, wherein the backhand piece is coupled to the palm piece such that the front and rear index finger portions are together configured to receive an index finger, that the front and rear middle finger portions are together configured to receive a middle finger, and that the front and rear shell portions are together configured to receive a third finger and a little finger; and
a thumb piece coupled to the thumbhole of the palm piece and configured to receive a thumb.

9. The welding glove of claim 8, wherein the front index finger portion, the front middle finger portion and the front shell portion are separately formed.

10. A four-fingered welding glove, comprising:
an integrated sheet having a front shell portion, a front index finger portion, a rear index finger portion, a rear middle finger portion, a rear shell portion and a thumbhole;
a front middle finger piece separately cut and sewn between the front shell portion and the front index finger portion, wherein the front and rear index finger portions are together configured to receive an index finger, wherein the front and rear middle finger portions are together configured to receive a middle finger, and wherein the front and rear shell portions are together configured to receive a third finger and a little finger; and
a thumb piece coupled to the thumbhole of the integrated sheet and configured to receive a thumb.

11. The welding glove of claim 10, wherein the front shell portion and the front index finger portion include a wing at a side surface thereof, respectively, and wherein the front middle finger piece is sewn between the wing of front shell portion and the wing of the front index finger portion.

12. The welding glove of claim 10, wherein the front index finger portion is adjacent to the rear index finger portion.

13. The welding glove of claim 10, wherein the front shell portion is adjacent to the rear shell portion.
